(19) United States
(12) Patent Application Publication
(10) Pub. No.: US 2011/0040095 A1
(43) Pub. Date: Feb. 17, 2011

(54) PREPARATION OF MONTELUKAST AND ITS SALTS

(75) Inventors: Satyanarayana Bollikonda, Hyderabad (IN); Kirankumar Venkata Kandirelli, Hyderabad (IN); Rama Krishna Venkata Medisetti, Visakhapatanam (IN); Janardana Sarma Ramachandra Kopparapu, Secunderabad (IN); Kushal Surajmal Manudhane, Hyderabad (IN)

Correspondence Address: DR. REDDY''S LABORATORIES, INC., 200 SOMERSET CORPORATE BLVD, SEVENTH FLOOR, BRIDGEWATER, NJ, 08807-2862, US

(73) Assignees: DR. REDDY'S LABORATORIES LTD., Hyderabad 500 016, Andhra Pradesh (IN); DR. REDDY'S LABORATORIES, INC., Bridgewater, NJ (US)

(21) Appl. No.: 12/921,618

(22) PCT Filed: Mar. 17, 2009
(86) PCT No.: PCT/US2009/037346

§ 371 Date: Sep. 9, 2010

Related U.S. Application Data
(60) Provisional application No. 61/073,837, filed on Jun. 19, 2008.

(30) Foreign Application Priority Data
Mar. 17, 2008 (IN) 655/CHE/2008

(51) Int. Cl. C07D 215/14

(52) U.S. Cl. 546/174

(57) ABSTRACT

Processes for preparing montelukast acid and its salts.

### INTRODUCTION

The present application relates to processes for preparing montelukast acid and its salts.

The drug compound having the adopted name “montelukast sodium” has a chemical name [R-(E)]-1-[[[1-[3-[2-(7-chloro-2-quinolinyl)ethenyl]phenyl]-3-[2-(1-hydroxy-1-methylethyl)phenyl]propyl]thio]methyl]cyclopropaneacetic acid, monosodium salt and is represented by structural Formula I.

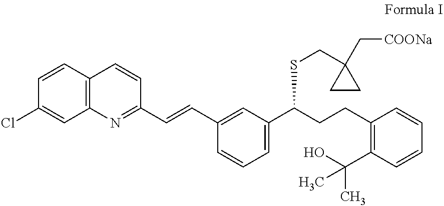

Montelukast sodium is a selective and orally active leukotriene receptor antagonist that inhibits the cysteinyl leukotriene CysLT₁ receptor and is useful in the treatment of asthma as well as other conditions mediated by leukotrienes, such as inflammation and allergies.

Montelukast sodium is commercially available in products sold under the trademark SINGULAIR. SINGULAIR tablets contain 4.2 mg, 5.2 mg and 10.4 mg of montelukast sodium, respectively, equivalent to 4 mg, and 5 mg and 10 mg of montelukast acid, respectively.

U.S. Pat. No. 5,565,473 discloses montelukast acid and its related compounds along with their pharmaceutically acceptable salts. It also provides processes for their preparation.

U.S. Pat. No. 5,614,632 discloses a process for preparation of montelukast acid, its intermediates and montelukast sodium. The process involves the condensation of a mesylated intermediate of Formula III

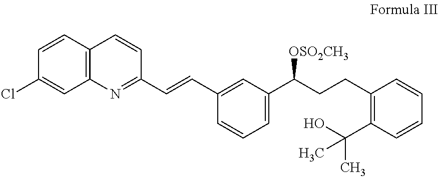

with the dilithium anion of 1-mercaptomethyl cyclopropaneacetic acid of Formula IV

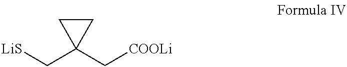

to obtain a reaction mixture containing montelukast in the form of its lithium salt, which is then converted into montelukast acid by treating the reaction mixture with a water soluble carboxylic acid such as acetic acid, oxalic acid or tartaric acid; followed by conversion of the resulting montelukast acid into the dicyclohexylamine salt of montelukast. It is purified and converted into montelukast acid by treating with a water soluble carboxylic acid such as acetic acid, oxalic acid or tartaric acid; and subsequently the resulting montelukast acid is converted into montelukast sodium by reacting with sodium hydroxide.

The above process suffers from major disadvantages of low yield and quality of montelukast acid, and subsequently of montelukast sodium, resulting in an uneconomical process.

### SUMMARY

The present application provides processes for preparing montelukast acid and its salts.

In an embodiment, there is provided a process for the preparation of montelukast acid of Formula II, or a salt thereof,

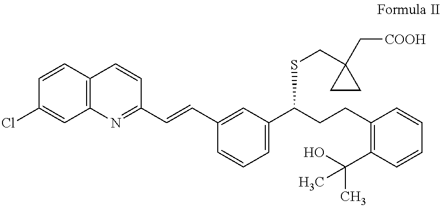

which process includes:

(a) providing a solution of a salt of montelukast;

(b) treating the solution with a salt breaking agent (hereinafter referred to as a “desalting agent”), to convert the salt into montelukast, with the proviso that the desalting agent is not a water-soluble organic acid;

(c) converting the montelukast into a salt;

(d) optionally purifying the salt of montelukast obtained in (c);

(e) treating the salt of montelukast from (c) or (d) with a desalting agent to convert the salt into montelukast acid; and

(f) optionally, converting the resulting montelukast acid into a salt.

In an embodiment, the present application provides pure montelukast free acid and its sodium salt.

### DETAILED DESCRIPTION

An aspect of the present application provides processes for preparing montelukast acid and its salts.

An embodiment provides a process for the preparation of montelukast acid of Formula II, or a salt thereof, including:

(a) providing a solution of a salt of montelukast;

(b) treating the solution of a salt of montelukast with a desalting agent to convert the salt of montelukast into montelukast, with the proviso that the desalting agent is not a water-soluble organic acid;

(c) converting the montelukast into a salt;

(d) optionally, purifying the salt of montelukast obtained in (c);

(e) treating the salt of (c) or (d) with a desalting agent to convert the salt into montelukast acid; and

(f) optionally, converting the montelukast acid into a salt.

Step (a) involves providing a solution of a salt of montelukast.

The salt of montelukast in (a) includes, but is not limited to, a metal salt and the like.

In (a), the solution of a salt of montelukast is provided for example, by dissolving a salt of montelukast in a suitable solvent. Alternatively, the solution may be obtained directly from a reaction mixture that is obtained, for example, by the reaction of a mesylated intermediate of Formula III,

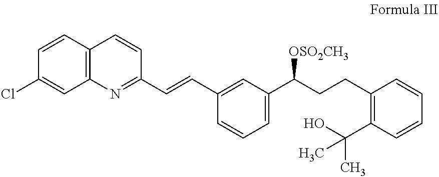

with 1-mercaptomethylcyclopraneacetic acid of Formula V,

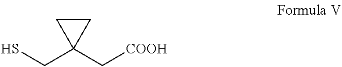

in the presence of a metal source.

Useful metal sources include, but are not limited to, alkali or alkaline earth metal sources, such as a lithium or sodium source.

A salt of montelukast may be prepared, for example, by a process disclosed in International Application No. PCT/US2007/083756, filed on Nov. 6, 2007, which is incorporated herein by reference in its entirety, or it may be prepared by other processes known in the art.

Suitable temperatures for addition of desalting agent into the mixture containing a salt of montelukast range from about −15° C. to about 45° C., or about 25° C. to about 35° C.

Suitable solvents which may be used in (a) include, but are not limited to, organic solvents such as: halogenated hydrocarbons such as dichloromethane, ethylene dichloride, chloroform, and the like; ketones, such as ethyl methyl ketone, methyl isobutyl ketone, and the like; hydrocarbon solvents such as toluene, xylene, n-hexane, n-heptane, cyclohexane and the like; esters such as ethyl acetate, n-propyl acetate, n-butyl acetate, t-butyl acetate, and the like; ethers such as diethyl ether, diisopropyl ether, methyl t-butyl ether, tetrahydrofuran, dioxane, and the like; polar aprotic solvents such as N,N-dimethylformamide, N,N-dimethylacetamide, dimethylsulphoxide, sulpholane, N-methylpyrrolidone, and the like; nitriles such as acetonitrile, propionitrile, and the like; and mixtures thereof or their combinations with water in various proportions.

Step (b) involves treating said solution of a salt of montelukast with a desalting agent to convert said salt of montelukast into montelukast, with the proviso that the desalting agent is not a water-soluble organic acid.

As set forth above, the prior processes describes such conversion with organic acids, for example, acetic acid, oxalic acid, and tartaric acid. In contrast, the present patent application describes the use of alternative desalting agents.

Suitable desalting agents that may be used in the present application include, but are not limited to: inorganic acids, such as hydrochloric acid, hydrobromic acid, hydroiodic acid, hydrofluoric acid, sulfuric acid, nitric acid, phosphoric acid, polyphosphoric acid, and the like; salts such as sodium dihydrogen phosphate, sodium bicarbonate, potassium dihydrogen phosphate, potassium bicarbonate, ammonium chloride, ammonium sulphate, ammonium bromide, ammonium phosphate, ammonium carbonate and the like; and resins such as cation exchange resins, anion exchange resins, chelated resins, and the like.

Suitably, aqueous solutions containing about 5% to about 50%, or about 10% to about 20%, (w/v) of the desalting agent may be used.

Polymer matrixes that may be used for the above resins include, but are not limited to, styrene-divinylbenzene, acrylic-divinylbenzene, phenolic, formophenolic, cellulose, agarose, polystyrene copolymer, crosslinked sephadex/dextran, crosslinked polystyrene, crosslinked cellulose, crosslinked agarose, and the like. Cation exchange resins include, but are not limited to, polymer matrixes with surface functional groups such as sulfonic acid, nitric acid, phosphonic acid, carboxylic acid, and the like. Anion exchange resins include, but are not limited to, polymer matrixes with surface functional groups such as quaternary ammonium, diethylaminoethyl, triethylamine, and related groups. Chelated resins include, but are not limited to, polymer matrixes with surface functional groups such as methylenethiol, imminodiacetic acid, N-methylglucamine, aminophosphonic, and related groups. Suitable exchange resins include resins having a styrene-divinylbenzene polymer matrix and an acidic functional group, such as the Tulsion® T63 (MP), Tulsion T5201R, and Tulsion T57 resins with nuclear sulphonic functional groups, all manufactured by Thermax Limited, Pune, India.

When a resin is used as a desalting agent, after completion of desalting, the resin may be optionally recovered by techniques such as filtration, centrifugation and the like; and it may be reused for a number of cycles and thus making the process more economical and ecologically friendly. Use of other desalting agents of the present application also make the process simple, economical and ecologically friendly, as compared to the prior methods where water-soluble organic acids are used.

Step (c) involves converting the montelukast into its salt.

The salt in (c) may be a salt with a suitable organic amine, or with a suitable metal ion. Suitable organic amines that may be used for preparation of an organic amine salt of montelukast include, but are not limited to, dicyclohexylamine, dipropylamine, diisopropylamine, α-methylbenzylamine, cyclohexylethylamine, t-butyl amine, and the like. Suitable metal ions with which salts of montelukast may be formed include, but are not limited to, lithium, sodium, potassium, cesium, magnesium, calcium, strontium, and the like. Suitable metal sources that may be used for preparation of a metal salt of montelukast include, but are not limited to, lithium hydroxide, sodium hydroxide, potassium hydroxide, calcium hydroxide, magnesium hydroxide, lithium carbonate, sodium carbonate, potassium carbonate, cesium carbonate, calcium carbonate, magnesium carbonate, sodium bicarbonate, potassium bicarbonate, sodium methoxide, potassium methoxide, sodium t-butoxide, potassium t-butoxide, and the like.

The conversion in (c) may be carried out in suitable solvents, which include but are not limited to: alcohols such as methanol, ethanol, 1-propanol, 2-propanol, 1-butanol, 2-butanol, t-butyl alcohol, 1-pentanol, 2-pentanol, neopentyl alcohol, amyl alcohol, 2-methoxyethanol, 2-ethoxyethanol, ethylene glycol, glycerol, and the like; ketones such as acetone, butanone, 2-pentanone, 3-pentanone, methyl butyl ketone, methyl ethyl ketone, methyl iso-butyl ketone and the like; esters such as ethyl formate, methyl acetate, ethyl acetate, propyl acetate, t-butyl acetate, isobutyl acetate, methyl propanoate, ethyl proponoate, methyl butanoate, ethyl butanoate and the like; ethers such as diethyl ether, diisopropyl ether, t-butyl methyl ether, dibutyl ether, tetrahydrofuran, 1,2-dimethoxyethane, 1,4-dioxane, 2-methoxyethanol, 2-ethoxyethanol, anisole and the like; aliphatic or alicyclic hydrocarbons such as hexanes, n-heptane, n-pentane, cyclohexane, methylcyclohexane, nitromethane and the like; chlorinated hydrocarbons such as dichloromethane, chloroform, 1,1,2-trichloroethane, 1,2-dichloroethene and the like; aromatic hydrocarbons such as toluene, xylenes, chlorobenzene, tetraline and the like; nitriles such as acetonitrile, propionitrile and the like; polar aprotic solvents such as N,N-dimethylformamide, N,N-dimethylacetamide, N-methylpyrrolidone, pyridine, dimethylsulphoxide, sulpholane, formamide, acetamide, propanamide and the like; water; and mixtures thereof.

The resulting salt in (c) may be isolated using techniques known in the art. For example, useful techniques include but are not limited to: decantation, centrifugation, gravity filtration, suction filtration, concentrating, cooling, stirring, shaking, adding an anti-solvent, adding seed crystals, evaporation, flash evaporation, simple evaporation, rotational drying, spray drying, thin-film drying, freeze-drying, and the like.

The resulting solid may optionally be washed with a suitable solvent to remove occluded mother liquor, in order to reduce amounts of the impurities entrapped in the wet cake. The so-obtained wet cake may be optionally dried by conventional drying techniques such as tray dryer, cone vacuum dryer, fludized bed dryer, agitated thin film dryer, and the like at atmospheric pressure or under reduced pressure.

Step (d) involves optionally purifying the salt of montelukast obtained in (c).

The salt of montelukast obtained in (c) may be prepared and purified by a process disclosed in International Application No. PCT/US2007/083756, filed on Nov. 6, 2007, or by any other processes known in the art.

Alternatively the salt of montelukast may be purified by a process, which includes:

i) providing a solution of salt of montelukast in a suitable solvent;

ii) isolating a pure salt of montelukast;

iii) optionally, drying the salt.

The solution in step i) of the above process may be obtained by dissolving a salt of montelukast in a suitable solvent, or it may be obtained directly from a reaction mixture that is obtained from synthesis of the compound.

Suitable solvents, which may be used in step i) include but are not limited to: alcohols such as methanol, ethanol, 1-propanol, 2-propanol, 1-butanol, 2-butanol, t-butyl alcohol, 1-pentanol, 2-pentanol, neopentyl alcohol, amyl alcohol, 2-methoxyethanol, 2-ethoxyethanol, ethylene glycol, glycerol and the like; ketones such as acetone, butanone, 2-pentanone, 3-pentanone, methyl butyl ketone, methyl ethyl ketone, methyl iso-butyl ketone and the like; esters such as ethyl formate, methyl acetate, ethyl acetate, propyl acetate, t-butyl acetate, isobutyl acetate, methyl propanoate, ethyl proponoate, methyl butanoate, ethyl butanoate and the like; ethers such as diethyl ether, diisopropyl ether, t-butyl methyl ether, dibutyl ether, tetrahydrofuran, 1,2-dimethoxyethane, 1,4-dioxane, 2-methoxyethanol, 2-ethoxyethanol, anisole and the like; aliphatic or alicyclic hydrocarbons such as hexanes, n-heptane, n-pentane, cyclohexane, methylcyclohexane, nitromethane and the like; chlorinated hydrocarbons such as dichloromethane, chloroform, 1,1,2-trichloroethane, 1,2-dichloroethene and the like; aromatic hydrocarbons such as toluene, xylenes, chlorobenzene, tetraline and the like; nitriles such as acetonitrile, propionitrile and the like; polar aprotic solvents such as N,N-dimethylformamide, N,N-dimethylacetamide, N-methylpyrrolidone, pyridine, dimethylsulphoxide, sulpholane, formamide, acetamide, propanamide and the like; water; and mixtures thereof.

The isolation in step (ii) may be effected by techniques including, but not limited to, crystallization, decantation, centrifugation, gravity filtration, suction filtration, concentrating, cooling, stirring, shaking, adding an anti-solvent, adding seed crystals, evaporation, flash evaporation, simple evaporation, rotational drying, spray drying, thin-film drying, freeze-drying, and the like.

The pure salt of montelukast obtained in step (d) may have a purity greater than about 98%, greater than about 99%, greater than about 99.5%, or greater than about 99.8%, by weight as determined using high performance liquid chromatography (HPLC).

Step (e) involves treating the purified salt of montelukast with a desalting agent to convert said salt to montelukast free acid.

Step (e) involves obtaining montelukast acid from the purified salt obtained in (d) involving desalting by treating the salt of montelukast, optionally in a suitable solvent, with a suitable desalting agent.

Suitable desalting agents for use in (e) include, but are not limited to, salts, organic acids, inorganic acids, resins, and the like.

Suitably aqueous solutions containing about 5% to about 50%, or about 10% to about 20%, (w/v) of the desalting agent may be used.

Suitable organic acids that may be used in (d) include, but are not limited to, acetic acid, oxalic acid, tartaric acid, n-propionic acid, isopropanoic acid, n-butyric acid, isobutyric acid, and the like.

Suitable inorganic acids that may be used in (d) include, but are not limited to, hydrochloric acid, hydrobromic acid, hydroiodic acid, hydrofluoric acid, sulfuric acid, nitric acid, phosphoric acid, polyphosphoric acid, and the like.

Suitable salts that may be used in (d) include, but are not limited to, sodium dihydrogen phosphate, ammonium chloride, ammonium sulphate, ammonium bromide, ammonium phosphate, ammonium carbonate, and the like.

Suitable resins that may be used in (d) include, but are not limited to, cation exchange resins, anion exchange resins, and chelated resins. Polymer matrixes that may be used for these resins include, but are not limited to styrene-divinylbenzene, acrylic-divinylbenzene, phenolic, formophenolic, cellulose, agarose, polystyrene copolymer, crosslinked sephadex/dextran, crosslinked polystyrene, crosslinked cellulose, crosslinked agarose, and the like. Cation exchange resins include, but are not limited to, polymer matrixes with surface functional groups such as sulfonic acid, nitric acid, phosphonic acid, carboxylic acid, and the like. Anion exchange resins include, but are not limited to, polymer matrixes with surface functional groups such as quaternary ammonium, diethylaminoethyl, triethylamine, and related groups. Chelated resins include, but are not limited to, polymer matrixes with surface functional groups such as methylenethiol, imminodiacetic acid, N-methylglucamine, aminophosphonic, and related groups. Suitable exchange resins include resins having a styrene-divinylbenzene polymer matrix and an acidic functional group, such as the Tulsion® T63 (MP), Tulsion T5201R, and Tulsion T57 resins with nuclear sulphonic functional groups, all manufactured by Thermax Limited, Pune, India.

When a resin is used as a desalting agent, after completion of desalting, the resin may be optionally recovered by techniques such as filtration, centrifugation and the like; and it may be reused for a number of cycles and thus make the process more economical and ecologically friendly. Use of other desalting agents of the present application also makes the process simple, economical and ecologically friendly, as compared to the prior methods where a water soluble organic acid is used for desalting.

Suitable temperatures for addition of desalting agent into a mixture containing a salt of montelukast range from about −15° C. to about 35° C., or about 25° C. to about 35° C.

Suitable solvents that may be used in e) include, but are not limited to: halogenated hydrocarbons such as dichloromethane, ethylene dichloride, chloroform, and the like; ketones, such as ethyl methyl ketone, methyl isobutyl ketone, and the like; hydrocarbons solvents such as toluene, xylene, n-hexane, n-heptane, cyclohexane and the like; esters such as ethyl acetate, n-propyl acetate, n-butyl acetate, t-butyl acetate, and the like; nitriles such as acetonitrile, propionitrile, and the like; and mixtures thereof or their combinations with water in various proportions.

Montelukast acid thus obtained may be further purified, if desired, by recrystallization or slurrying in a suitable solvent.

Suitable solvents which may be used for purifying montelukast acid include but are not limited to: alcohols such as methanol, ethanol, isopropyl alcohol, n-propanol, and the like; ketones such as acetone, ethyl methyl ketone, methyl isobutyl ketone and the like; ethers such as diethyl ether, diisopropyl ether, methyl t-butyl ether, tetrahydrofuran, dioxane, and the like; ethyl acetate, n-propyl acetate, n-butyl acetate, t-butyl acetate, and the like; nitriles such as acetonitrile, propionitrile, and the like; aprotic polar solvents such as N,N-dimethylformamide, N,N-dimethylacetamide, dimethylsulphoxide, sulpholane, N-methylpyrrolidone, and the like; hydrocarbons such as toluene, xylene, n-heptane, cyclohexane, and the like; and mixtures thereof or their combinations with water in various proportions.

Step (f) involves optionally converting the resulting montelukast acid into its salt.

An optional conversion of montelukast acid into its salt, such as a sodium salt, can be accomplished using any processes known in the art, e.g., by a process disclosed in International Application No. PCT/US2007/083756, filed on Nov. 6, 2007.

In yet another embodiment, the present application provides pure montelukast free acid, or its salt, obtained from a process of the present application.

Montelukast free acid and its sodium salt has a purity greater than about 99%, greater than about 99.2%, greater than about 99.5, or greater than about 99.7%, by weight as determined using HPLC. The pure montelukast acid or its salt can contain at least one of the following impurities at concentrations less than about 0.5%, less than about 0.3%, less than about 0.2%, or less than about 0.1%, by weight as determined using HPLC:

A “sulphoxide” impurity having a chemical name 2-(1-{(1R)-1-{3-[(E)2-(7-chloro-2-quinolyl)-1-ethenyl]phenyl}-3-[2-(1-hydroxy-1-methylethyl)phenyl]propyl]sulfinylmethyl}cyclopropyl]acetic acid, and structural formula A.

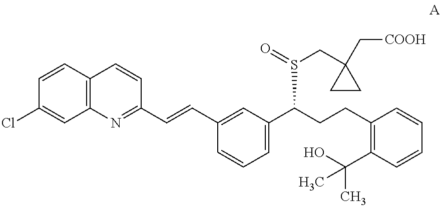

A “keto” impurity having a chemical name [R-(E)]-1-[[[1-[3-[2-(7-chloro-2-quinolinyl)ethenyl]phenyl]-3-[2-(methyloxo)phenyl]propyl]thio]methyl]cyclopropane acetic acid, and structural formula B.

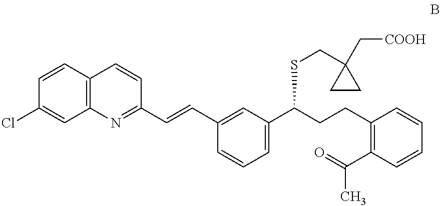

A “styrene” impurity having a chemical name [R-(E)]-1-[[[l-[3-[2-(7-chloro-2-quinolinyl)ethenyl]phenyl]-3-[2-(I-isopropenyl)phenyl]propyl]thio]methyl]cyclopropaneacetic acid, and structural formula C.

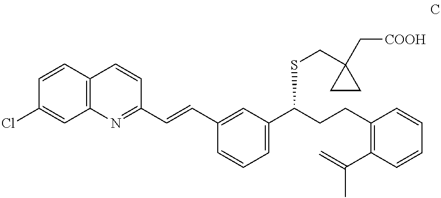

A “diol” impurity having a chemical name 1-{3-[2-(7-chloroquinolin-2-yl)-ethenyl]phenyl}-3-[2-(1-hydroxy-1-methylethyl)phenyl]propan-1-ol, and structural formula D.

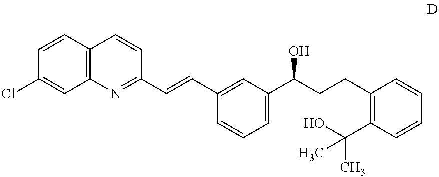

The purity of montelukast free acid of Formula (II) and its salts may be analyzed using high performance liquid chromatography (HPLC), for example by a method using a Hypersil C-18 column, 100×4.6 mm ID, 3 μm particle size, or equivalent. The other parameters of the method are as shown in Table 1.

TABLE 1

| | |
|---|---|
| Flow rate | 1.0 mL/minute. |
| Wavelength | 225 nm. |
| Injection load | 20.0 μL. |
| Temperature | Ambient. |
| Elution | Gradient. |
| Diluent | Methanol. |
| Sample preparation | Weigh a sample providing about 5 mg of |
| | montelukast, dissolve and dilute to 10 mL with |
| | diluent. |
| Mobile phase | Buffer: 3.7 g of sodium dihydrogen phosphate in |
| preparation | 1000 mL of water. pH adjusted to 3.7 with |
| | orthophosphoric acid. |
| | Mobile Phase A: mix buffer with acetonitrile in a |
| | volume ratio of 80:20. |
| | Mobile Phase B: mix buffer with acetonitrile in a |
| | volume ratio of 20:80. |

Certain specific aspects and embodiments of this invention are described in further detail by the examples below, which are provided only for the purpose of illustration and are not intended to limit the scope of the invention in any manner. In the examples, percentages are expressed by weight, unless the context indicates otherwise.

### Reference Example

### Preparation of Montelukast Acid (Formula II)

2-(2-(3-(S)-(3-(2-(7-chloro-2-quinolinyl)ethenyl)phenyl)-3-hydroxypropyl)phenyl)-2-propanol (a diol intermediate) (140 g) and toluene (700 mL) were charged into a round bottom flask and acetonitrile (1290 mL) was charged, followed by cooling to about −15±2.5° C. Diisopropylethylamine (69.5 mL) was charged followed by stirring for about 30 minutes. Methanesulfonyl chloride (26 mL) was added dropwise over about 30 minutes followed by stirring for about 9 hours. The formed solid was filtered and washed with acetonitrile (280 mL), followed by washing with chilled hexane (280 mL) to afford a mesylated compound of Formula III.

(1-Mercaptomethyl-cyclopropyl)acetic acid (67 g) and tetrahydrofuran (2100 mL) were charged into a clean and dry round bottom flask and cooled to about −12.5±2.5° C. n-Butyllithium in n-hexane (560 mL, 15% w/v) was added dropwise over about 30 minutes under an inert atmosphere. The reaction mass was stirred at −12.5±2.5° C. for about 30 minutes, followed by charging the above-obtained mesylated compound of Formula III (59 g) under an inert atmosphere. The mixture was stirred for about 6 hours at −2.5±2.5° C., followed by quenching the reaction mass by the addition of saturated sodium chloride solution (840 mL) at 2.5±7.5° C. The mixture was allowed to reach a temperature of about 30° C., and the organic and aqueous layers were separated. The organic layer was washed with a 40% w/v aqueous solution of ammonium chloride (1680 mL) at 26° C. followed by washing with water (2×560 mL) to obtain about 2110 ml of organic layer containing montelukast free acid, corresponding to 140 g of the diol intermediate.

### Example 1

### Preparation of Montelukast Acid Using Resin as a Desalting Agent

### (A) Preparation of Dicyclohexylamine Salt of Montelukast

An organic layer obtained by the procedure described in the Reference Example, starting from 30 g of 2-(2-(3(S)-(3-(2-(7-chloro-2-quinolinyl)ethenyl)phenyl)-3-hydroxypropyl)phenyl)-2-propanol, was charged into a round bottom flask followed by addition of dichloromethane (450 mL). Styrene-divinylbenzenesulfonic acid resin (50 g) and water (240 mL) were charged, stirred for about 30 minutes, and the resin was removed by filtration. The organic and aqueous layers were separated followed by washing the organic layer with water (2×240 mL). The organic layer was distilled completely at about 50° C. under reduced pressure. To the obtained residue, acetonitrile (2×48 mL) was added and distilled completely to remove traces of dichloromethane. The obtained residue was dissolved in acetonitrile (225 mL) and isopropanol (90 mL) at about 30±5° C. Dicyclohexylamine (17 mL) was added and the mixture was heated to reflux and stirred for about 15 minutes. The resulting solution was cooled to about 30±5° C. and stirred for about 8 hours. The separated solid was filtered, washed with acetonitrile (60 mL) and suction dried. The wet compound was dissolved in acetonitrile (225 mL) and isopropanol (90 mL) and heated to about 80° C. Carbon (3 g) was charged. The hot mixture was filtered through a Hyflow (flux-calcined diatomaceous earth) bed and was washed with mixture of acetonitrile (45 mL) and isopropanol (15 mL). The filtrate was stirred at 27° C. until solid formation was complete. The solid was filtered, washed with acetonitrile (60 mL) and suction dried to afford a dicyclohexylamine salt of montelukast.

### (B) Preparation of Montelukast Acid

The wet compound obtained from (A) was charged into a round bottom flask followed by addition of dichloromethane (300 mL) and stirred for about 30 minutes. Glacial acetic acid (5.7 mL) and water (240 mL) were charged and stirred for about 30 minutes at about 30±5° C. The organic and aqueous layers were separated. The aqueous layer was extracted with dichloromethane (300 mL). Organic layers were combined followed by washing with water (2×240 mL). The organic layer was distilled completely at about 50° C. The obtained residue was dissolved in methanol (60 mL) and distilled at below 55° C. to remove trace of dichloromethane. Methanol (24 mL) was added to the residue and stirred at 50±5° C. for 45 minutes. The mass was cooled to 30±5° C. and stirred for 5 hours, then was further cooled to 0±5° C. and stirred for 4 hours. The formed solid was filtered, washed with chilled methanol (7.5 mL) and dried at about 50±5° C. under reduced pressure to afford the title compound. Yield 16.9 g, purity by HPLC 99.67%.

### Example 2

### Preparation of Montelukast Acid Using Resin as a Desalting Agent

### (A) Preparation of Dicyclohexylamine Salt of Montelukast

An organic layer obtained by the procedure described in the Reference Example, prior to desalting, starting from 16 g of 2-(2-(3-(S)-(3-(2-(7-chloro-2-quinolinyl)ethenyl)phenyl)-3-hydroxypropyl)phenyl)-2-propanol, was charged into a round bottom flask followed by addition of dichloromethane (240 mL), and the mixture was stirred for about 10 minutes. TULSION T63 resin (27 mL) and water (128 mL) were charged and stirred for about 30 minutes at about 30±5° C. and the resin was removed by filtration. The filtrate was washed with dichloromethane (32 mL). The organic and aqueous layers were separated, followed by washing the organic layer with water (2×128 mL). The organic layer was distilled completely at about 50° C. under reduced pressure. To the obtained residue, acetonitrile (2×48 mL) was added and distilled completely to remove the traces of dichloromethane. The obtained residue was dissolved in acetonitrile (120 mL) and stirred for about 5 minutes at 48±2° C. followed by addition of isopropanol (48 mL). Dicyclohexylamine (9 mL) was added and the mixture was heated to reflux and stirred for about 15 minutes. The resulting solution was cooled to about 30±5° C. and stirred for about 9 hours. The reaction mass was then filtered, washed with acetonitrile (32 mL) and suction dried. The wet compound was dissolved in acetonitrile (120 mL) and isopropanol (48 mL) and heated to about 80° C. Carbon (1.6 g) was charged. The hot mixture was filtered through a Hyflow bed and washed with acetonitrile (24 mL) and isopropanol (8 mL). The filtrate was stirred at 28° C. until solid formation was complete. The solid was filtered, washed with acetonitrile (32 mL) and suction dried to afford a dicyclohexylamine salt of montelukast.

### (B) Preparation of Montelukast Acid

The wet compound obtained from (A) was charged into a round bottom flask followed by addition of dichloromethane (160 mL) and stirred for about 15 minutes. Styrene-divinylbenzenesulfonic acid resin (40 mL) and water (160 mL) were charged and stirred for about 30 minutes at about 30±5° C. and the resin was filtered. The filtrate was washed with dichloromethane (32 mL). The organic and aqueous layers were separated. The aqueous layer was extracted with dichloromethane (160 mL). Organic layers were combined followed by washing with water (2×128 mL). The organic layer was distilled completely at about 50° C. The obtained residue was dissolved in methanol (32 mL) and the solution distilled at about 50±5° C. to remove traces of dichloromethane. The residue was dissolved in methanol (16 mL), followed by stirring at 30±5° C. for 4-6 hours and then at 2.5±2.5° C. for 4 hours. The formed solid was filtered and washed with chilled methanol (5 mL). The solid was dried under reduced pressure at 50±5° C. to afford the title compound. Yield 7.6 g, chemical purity by HPLC 99.59%, stryrene impurity 0.02%, diol impurity 0.13%.

### Example 3

### Preparation of Montelukast Acid Using Ammonium Chloride as a Desalting Agent

### (A) Preparation of Dicyclohexylamine Salt of Montelukast

An organic layer obtained by the procedure described in the Reference Example, prior to desalting, starting from 30 g of 2-(2-(3-(S)-(3-(2-(7-chloro-2-quinolinyl)ethenyl)phenyl)-3-hydroxypropyl)phenyl)-2-propanol, was charged into a round bottom flask followed by addition of dichloromethane (450 mL) and stirred for about 10 minutes. Saturated ammonium chloride solution (240 mL) was charged slowly and stirred for about 30 minutes. The organic and aqueous layers were separated, followed by washing the organic layer with water (2×240 mL). The organic layer was distilled completely at about 50° C. To the obtained residue, acetonitrile (2×90 mL) was added and distilled completely to remove traces of dichlormethane. The obtained residue was dissolved in acetonitrile (225 mL) and isopropanol (90 mL) at about 25° C. Dicyclohexylamine (15.43 g) was added and the mixture was heated to about 80° C. and stirred for about 60 minutes. The mixture was cooled to about 35° C. and stirred for solid formation. The solid was filtered, washed with acetonitrile (60 mL) and suction dried. The wet compound was dissolved in acetonitrile (225 mL) and isopropanol (90 mL) and heated to about 80° C. Carbon (3 g) was charged to the solution. The solution was filtered through a Hyflow bed and washed with acetonitrile (45 mL) and isopropanol (15 mL). The filtrate was stirred at 29° C. for solid formation. The solid was filtered, washed with acetonitrile (60 mL) and suction dried to afford a dicyclohexylamine salt of montelukast. Chemical purity by HPLC 99.56%, sulphoxide impurity 0.04%, keto impurity 0.04%, stryrene impurity 0.03%, diol impurity 0.08%.

### (B) Preparation of Montelukast Acid

The wet compound obtained from (A) was charged into a round bottom flask followed by addition of dichloromethane (300 mL) and stirred for about 15 minutes. Acetic acid (5.7 mL) and water (240 mL) were charged and stirred for about 15 minutes at about 30±5° C. The organic and aqueous layers were separated. The aqueous layer was extracted with dichloromethane (300 mL). Organic layers were combined followed by washing with water (2×240 mL). The organic layer was distilled completely at about 50° C. The obtained residue was dissolved in methanol (60 mL) and distilled below 55° C. to remove traces of dichloromethane. The residue was cooled to 30±5° C. and methanol (24 mL) was added. The mixture was stirred at 30±5° C. for about 5 hours and then further cooled to 0±5° C. and stirred for about 5 hours. The formed solid was filtered, washed with chilled methanol (7.5 mL) and dried at 55±5° C. under reduced pressure to afford the title compound. Yield 16.8 g, chemical purity by HPLC 99.69%, keto impurity 0.04%.

### Example 4

### Preparation of Montelukast Acid Using Sodium Dihydrogen Phosphate Monohydrate as a Desalting Agent

### (A) Preparation of Dicyclohexylamine Salt of Montelukast

An organic layer obtained by the procedure described in the Reference Example, prior to desalting, starting from 30 g of 2-(2-(3-(S)-(3-(2-(7-chloro-2-quinolinyl)ethenyl)phenyl)-3-hydroxypropyl)phenyl)-2-propanol, was charged into a round bottom flask followed by addition of dichloromethane (450 mL). Sodium dihydrogen phosphate (22.6 g) dissolved in water (240 mL) was charged and stirred for about 30 minutes at 30±5° C. The organic and aqueous layers were separated, followed by washing the organic layer with water (2±240 mL). The organic layer was distilled completely at about 50° C. To the obtained residue, acetonitrile (2×90 mL) was added and distilled completely to remove traces of dichloromethane. The obtained residue was dissolved in acetonitrile (225 mL) and isopropanol (90 mL) at about 30±5° C. Dicyclohexylamine (15.43 g) was added and the mixture heated to about 80° C. and stirred for about 15-30 minutes. The mixture was cooled to 30±5° C. and stirred for solid formation. The solid was filtered, washed with acetonitrile (60 mL) and sution dried. The wet compound was dissolved in acetonitrile (225 mL) and isopropanol (90 mL) and heated to reflux temperature. Carbon (3 g) was charged to the solution, then was filtered through a Hyflow bed and washed with acetonitrile (45 mL) and isopropanol (15 mL). The filtrate was stirred at 30±5° C. for about 6 hours for solid formation. The solid was filtered, washed with acetonitrile (60 mL) and suction dried to afford a dicyclohexylamine salt of montelukast. Chemical purity by HPLC 99.60%, sulphoxide impurity 0.05%, keto impurity 0.04%, stryrene impurity 0.03%.

### (B) Preparation of Montelukast Acid

The wet compound obtained from (A) was charged into a round bottom flask followed by addition of dichloromethane (300 mL). Acetic acid (5.7 mL) and water (240 mL) were charged and stirred for about 30 minutes at about 30±5° C. The organic and aqueous layers were separated. The aqueous layer was extracted with dichloromethane (300 mL). Organic layers were combined, followed by washing with water (2×240 mL). The organic layer was distilled completely at about 50° C. The obtained residue was dissolved in methanol (60 mL) and distilled below 55° C. to remove traces of dichloromethane. The residue was cooled to about 30±5° C. and methanol (24 mL) was added, followed by stirring at 30±5° C. for about 6 hours, and then at 2.5±2.5° C. for about 4 hours. The formed solid was filtered, washed with chilled methanol (7.5 mL), and dried at 55±5° C. under reduced pressure to afford the title compound. Yield 17.0 g, chemical purity by HPLC 99.68%, diol impurity 0.04%).

### Example 5

### Preparation of Montelukast Acid Using Sulphuric Acid as a Desalting Agent

### (A) Preparation of Dicyclohexylamine Salt of Montelukast

An organic layer obtained by the procedure described in the Reference Example, prior to desalting, starting from 8 g of 2-(2-(3-(S)-(3-(2-(7-chloro-2-quinolinyl)ethenyl)phenyl)-3-hydroxypropyl)phenyl)-2-propanol, was charged into a round bottom flask followed by addition of dichloromethane (120 mL). Sulphuric acid (0.51 g) dissolved in chilled water (64 mL) was charged and stirred for about 30 minutes. The organic and aqueous layers were separated, followed by washing the organic layer with water (2×64 mL). The organic layer was distilled completely at about 50° C. To the obtained residue, acetonitrile (2×24 mL) was added and distilled completely to remove traces of dichloromethane. The obtained residue was dissolved in acetonitrile (60 mL) and isopropanol (24 mL) at about 30±5° C. Dicyclohexylamine (4.6 g) was added and the mixture was heated to about 80° C. and stirred for about 60 minutes. The mixture was cooled to about 30±5° C. and stirred for solid formation. The solid was filtered, washed with acetonitrile (16 mL) and suction dried. The wet compound was dissolved in acetonitrile (60 mL) and isopropanol (24 mL) and heated to about 80° C. Carbon (0.8 g) was charged to the solution, then was filtered through a Hyflow bed and washed with acetonitrile (12 mL) and isopropanol (4 mL). The filtrate was stirred at 30±5° C. for about 8 hours for solid formation. The solid was filtered, washed with acetonitrile (16 mL) and suction dried to afford a dicyclohexylamine salt of montelukast. Chemical purity by HPLC 99.64%, sulphoxide impurity 0.10%, keto impurity 0.05%, stryrene impurity 0.09%.

### (B) Preparation of Montelukast Acid

The wet compound obtained from (A) was charged into a round bottom flask, followed by addition of dichloromethane (80 mL) and was stirred for about 10 minutes. Acetic acid (1.5 mL) and water (64 mL) were charged and stirred for about 30 minutes at about 30±5° C. The organic and aqueous layers were separated. The aqueous layer was extracted with dichloromethane (80 mL). Organic layers were combined followed by washing with water (2×64 mL). The organic layer was distilled completely at about 50° C. The obtained residue was dissolved in methanol (16 mL) and distilled below 55° C. to remove traces of dichloromethane. The residue was cooled to about 30±5° C. and methanol (6.4 mL) was added, followed by stirring at 30±5° C. for about 4 hours and then at 2.5±2.5° C. for about 6 hours. The solid was filtered, washed with chilled methanol (2 mL) and dried at 55±5° C. under reduced pressure to afford the title compound. Yield 4.5 g, chemical purity by HPLC 99.64%, sulphoxide impurity 0.06%, stryrene impurity 0.10%.

### Example 6

### Preparation of Montelukast Acid Using Hydrochloric Acid as a Desalting Agent

### (A) Preparation of Dicyclohexylamine Salt of Montelukast

An organic layer obtained by the procedure described in the Reference Example, prior to desalting, starting from 8 g of 2-(2-(3-(S)-(3-(2-(7-chloro-2-quinolinyl)ethenyl)phenyl)-3-hydroxypropyl)phenyl)-2-propanol, was charged into a round bottom flask followed by addition of dichloromethane (120 mL) and stirring for about 10 minutes. Hydrochloric acid (4.46 g) dissolved in water (64 mL) was charged and stirred for about 15-30 minutes. The organic and aqueous layers were separated followed by washing the organic layer with water (2×64 mL). The organic layer was distilled completely at about 50° C. To the obtained residue, acetonitrile (2×24 mL) was added and distilled completely to remove traces of dichloromethane. The obtained residue was dissolved in acetonitrile (60 mL) and isopropanol (24 mL) at about 30±5° C. Dicyclohexylamine (4.6 g) was added and the mixture was heated to about 80° C. and stirred for about 60 minutes. The mixture was cooled to about 30±5° C. and stirred for solid formation. The solid was filtered, washed with acetonitrile (16 mL) and suction dried. The wet compound was dissolved in acetonitrile (60 mL) and isopropanol (24 mL) and heated to about 80° C. Carbon (0.8 g) was charged to the solution, then was filtered through a Hyflow bed and washed with acetonitrile (12 mL) and isopropanol (4 mL). The filtrate was stirred at 30±5° C. for about 8 hours for solid formation. The solid was filtered, washed with acetonitrile (16 mL) and suction dried to afford a dicyclohexylamine salt of montelukast.

### (B) Preparation of Montelukast Acid

The wet compound obtained from (A) was charged into a round bottom flask, followed by addition of dichloromethane (80 mL) and stirring for about 10 minutes. Acetic acid (1.5 mL) and water (64 mL) were charged and stirred for about 30 minutes at about 30±5° C. The organic and aqueous layers were separated. The aqueous layer was extracted with dichloromethane (80 mL). Organic layers were combined followed by washing with water (2×64 mL). The organic layer was distilled completely at about 50° C. The obtained residue was dissolved in methanol (16 mL) and distilled below 55° C. to remove traces of dichloromethane. The residue was cooled to about 30±5° C. and methanol (6.4 mL) was added, followed by stirring at 30±5° C. for about 4 hours and then at 2.5±2.5° C. for about 6 hours. The formed solid was filtered, washed with chilled methanol (2 mL) and dried at 55±5° C. under reduced pressure to afford the title compound. Yield 4.8 g, chemical purity by HPLC 99.72%, sulphoxide impurity 0.04%, stryrene impurity 0.067%.

### Example 7

### Preparation of a Salt of Montelukast Acid

(A) An organic layer obtained using the procedure of the Reference Example (450 mL) was distilled completely at about 50° C. To the obtained residue, toluene (60 mL) was added and distilled completely. The obtained residue was dissolved in toluene (120 mL). The solution of about 150 ml is divided into two equal parts, each part equivalent to a 15 g batch size of diol for the preparation of a salt of montelukast.

(B) Preparation of Montelukast Acid Via a T-Butylamine Salt of Montelukast

To a solution of montelukast acid in toluene (75 mL) obtained in (A), t-butylamine (1.5 mL) was added at about 29° C. The mixture was seeded with a t-butyl amine salt of montelukast (0.2 g) and stirred for about 21 hours about 28° C. The formed solid was filtered, washed with toluene (10 mL) and suction dried. The wet compound was dried at about 51° C. under reduced pressure for about 3.5 hours to afford a crude t-butylamine salt of montelukast (4.5 g).

The crude t-butyl amine salt of montelukast (4.0 g) and toluene (32 mL) were charged into a round bottom flask, heated to about 70° C. and stirred for about 45 minutes. The mixture was filtered through s Hyflow bed and washed with toluene (8 mL). Isopropanol (1 mL) was added to the filtrate and was stirred at 30° C. for about 5.5 hours for solid formation. The solid was filtered, washed with toluene (8 mL) and suction dried. The wet compound was dried at about 55° C. for about 1 hour, 45 minutes to afford pure t-butylamine salt of montelukast. Yield 2.7 g, chemical purity by HPLC 99.50%, sulphoxide impurity 0.10%, keto impurity 0.04%, stryrene impurity 0.03%, diol impurity 0.02%.

The t-butylamine salt of montelukast (2.5 g) obtained above was charged into a round bottom flask followed by addition of dichloromethane (25 mL) at about 28° C. and stirred for about 5 minutes. Acetic acid (0.341 g) and water (12.5 mL) were charged and stirred for about 15 minutes at about 30±5° C. The organic and aqueous layers were separated and the organic layer was washed with water (3×12.5 mL). The organic layer was distilled completely at about 50° C. The obtained residue was dissolved in methanol (2.5 mL) and distilled completely at about 47° C. The residue was cooled to 30±5° C. and methanol (5 mL) was added. The mixture was stirred at 30±5° C. for about 90 minutes and then further cooled to about 5° C. and stirred for about 80 minutes. The formed solid was filtered, washed with chilled methanol (2.5 mL) and dried at 55±5° C. for about 3 hours to afford the title compound. Yield 1.7 g, chemical purity by HPLC 99.66%, sulphoxide impurity 0.07%, keto impurity 0.02%, stryrene impurity 0.04%.

(C) Preparation of Dicyclohexylamine Salt of Montelukast

To the solution of montelukast acid in toluene (75 ml) obtained in step (A), dicyclohexylamine (8.5 mL) was added and stirred at 26° C. until complete solid formation. The solid was filtered and washed with toluene. The wet compound was dried at about 50° C. under reduced pressure.

### Example 8

### Preparation of a Salt of Montelukast Acid

An organic layer obtained by the procedure described in the Reference Example (75 mL, equivalent to a batch size of 5 g of 2-(2-(3-(S)-(3-(2-(7-chloro-2-quinolinyl)ethenyl)phenyl)-3-hydroxypropyl)phenyl)-2-propanol) was distilled completely at about 50° C. To the obtained residue, toluene (10 mL) was added and distilled completely. The residue was dissolved in toluene (20 mL) and isopropyl alcohol (0.25 mL). Dicyclohexylamine (2.8 mL) was added at 26° C. and the mixture was stirred at 26° C. until complete solid formation. The solid was filtered and washed with a mixture of toluene and isopropyl alcohol and suction dried. The solid was dried at 60° C. under reduced pressure to afford 4.2 g of a pure dicyclohexylamine salt of montelukast. Chemical purity by HPLC 99.10%, sulphoxide impurity 0.168%, diol impurity 0.183, keto impurity 0.07%, stryrene impurity 0.086%.

The impurity levels can be further reduced by recrystallizing the compound from a mixture of toluene and isopropyl alcohol.

### Example 9

### Preparation of Montelukast Acid Using Ammonium Chloride as a Desalting Agent

### (A) Preparation of Dicyclohexylamine Salt of Montelukast

Saturated ammonium chloride solution (600 mL) was added to an organic layer obtained by the procedure described in the Reference Example, prior to desalting, starting from 50 g of 2-(2-(3-(S)-(3-(2-(7-chloro-2-quinolinyl)ethenyl)phenyl)-3-hydroxypropyl)phenyl)-2-propanol and stirred at about 26° C. for about 2 hours. The organic and aqueous layers were separated followed by washing the organic layer with water (2×200 mL). The organic layer was distilled completely at about 50° C. To the residue (˜10 g), toluene (20 mL) was added and distilled completely. The obtained residue was dissolved in toluene (40 mL) at about 25° C. Dicyclohexylamine (5.2 g) was added and stirred for about 3 hours. The mixture was seeded with a dicyclohexylamine salt of montelukast (0.1 g) and stirred for about 90 minutes. Toluene (40 mL) and isopropanol (0.5 mL) were added and stirred for about 15.5 hours. The formed solid was filtered, washed with a solution of toluene (25 mL) and isopropanol (0.32 mL) and suction dried. The wet compound was dried at about 60° C. for about 5.5 hours to afford a dicyclohexylamine salt of montelukast (8.1 g).

### (B) Preparation of Montelukast Acid

A dicyclohexylamine salt of montelukast (7.5 g) obtained from (A) was charged into a round bottom flask, followed by addition of dichloromethane (75 mL) and stirring for about 10 minutes. Acetic acid (1.96 g) and water (60 mL) were charged and stirred for about 15 minutes at about 30±5° C. The organic and aqueous layers were separated, and the organic layer was washed with water (2×60 mL). The organic layer was distilled completely at about 50° C. The obtained residue was dissolved in methanol (15 mL) and distilled at about 50° C. to remove traces of dichloromethane. The residue was cooled to 30±5° C. and methanol (7.5 mL) was added. The mixture was stirred at 30±5° C. for about 4 hours and then further cooled to 0±5° C. and stirred for about 4 hours. The compound was filtered, washed with chilled methanol (1.9 mL) and dried at about 50° C. under reduced pressure to afford title compound). Yield 4.6 g, chemical purity by HPLC 99.60%, sulphoxide impurity 0.03%, stryrene impurity 0.057%, keto impurity 0.07%.

### Example 10

### Preparation of Montelukast Sodium (Formula I)

Sodium hydroxide pellets (0.34 g) were dissolved in methanol (25 mL) and stirred for about 30 minutes. Montelukast acid (5 g) obtained by the procedure of Example 2 was charged into a round bottom flask followed by addition of methanol (25 mL) and stirring for about 15 minutes. The sodium hydroxide and montelukast acid solutions were combined and stirred for about 30 minutes at 30±5° C. Carbon (0.5 g) was charged and the mixture was filtered through a Hyflow bed and washed with methanol (10 mL). The filtrate was distilled completely under reduced pressure at a temperature of about 50° C. and the residue was dried at about 70° C. for 4 hours to yield the title compound. Yield 4.6 g, chemical purity by HPLC 99.7%, sulphoxide impurity 0.03%, stryrene impurity 0.057%, keto impurity 0.07%, chiral purity by HPLC 99.9%.

### Example 11

### Preparation of Montelukast Acid (Formula II)

2-(2-(3-(S)-(3-(2-(7-Chloro-2-quinolinyl)ethenyl)phenyl)-3-hydroxypropyl)phenyl)-2-propanol (30 g) and toluene (150 mL) were charged into a round bottom flask and acetonitrile (276 mL) was added, followed by cooling to about −15±5° C. Diisopropylethylamine (14.9 mL) was added, followed by stirring for about 30 minutes. Methanesulfonyl chloride (5.6 mL) was added dropwise, followed by stirring for about 9 hours. The formed solid was filtered and washed with chilled acetonitrile (60 mL), followed by washing with chilled hexanes (60 mL) and suction drying to afford a mesylated compound.

Dimethylsulfoxide (150 mL) and (1-mercaptomethylcyclopropyl) acetic acid (14.4 g) were charged into a flask and stirred for about 10 minutes. Sodium methoxide (39.1 mL, 28% w/w) solution in methanol was added and stirred for about 10 minutes. The mixture was cooled to −2.5±2.5° C. The mesylated compound obtained above was added slowly at about −5° C. The mixture was stirred for about 3 hours followed by quenching the reaction mass by the addition of saturated sodium chloride solution (180 mL) over about 30 minutes. The mixture was divided into three parts and processed as discussed below, using either sodium dihydrogen phosphate, ammonium chloride, or TULSION T-63 resin, respectively as desalting agents to afford a dicyclohexylamine salt of montelukast, and subsequently montelukast free acid, according to processes described in the previous examples.

(A): Sodium Dihydrogen Phosphate as Desalting Agent

Chemical purity of dicyclohexylamine salt of montelukast 99.17%, sulphoxide impurity 0.15%, diol impurity 0.10%, keto impurity 0.01%, styrene impurity 0.07%.

Chemical purity of montelukast free acid 99.48%, sulphoxide impurity 0.10%, styrene impurity 0.08%.

(B): Ammonium Chloride as Desalting Agent

Chemical purity of dicyclohexylamine salt of montelukast 99.68%, sulphoxide impurity 0.05%, diol impurity 0.03%, keto impurity 0.01%, styrene impurity 0.07%.

Chemical purity of montelukast free acid 99.62%, sulphoxide impurity 0.09%, styrene impurity 0.08%.

(C): Resin as Desalting Agent

Chemical purity of dicyclohexylamine salt of montelukast 99.08%, sulphoxide impurity 0.07%, diol impurity 0.11%, styrene impurity 0.08%.

Chemical purity of montelukast free acid 99.57%, sulphoxide impurity 0.08%, styrene impurity 0.08%.

1. A process for preparing montelukast acid or a salt thereof, comprising
(a) providing a solution of a salt of montelukast;
(b) treating the solution of (a) with a desalting agent to form montelukast, with the proviso that the desalting agent is not a water soluble organic acid;
(c) converting montelukast into a salt;
(d) optionally, purifying the salt of (c);
(e) treating a salt of (c) or (d) with a desalting agent to form montelukast; and
(f) optionally, converting the montelukast of (e) into a salt.

2. The process according to claim 1, wherein a salt of montelukast in (a) is a metal salt.

3. The process according to claim 1, wherein a salt of montelukast in (a) is an alkali metal or alkaline earth metal salt.

4. The process according to claim 1, wherein a salt of montelukast in (a) is a lithium or sodium salt.

5. The process according to claim 1, wherein a desalting agent in (b) comprises an inorganic acid, a salt, or a resin.

6. The process according to claim 1, wherein a desalting agent in (b) comprises at least one of hydrochloric acid, hydrobromic acid, hydroiodic acid, hydrofluoric acid, sulfuric acid, nitric acid, phosphoric acid, and polyphosphoric acid.

7. The process according to claim 1, wherein a desalting agent in (b) comprises an aqueous solution of an inorganic acid.

8. The process according to claim 1, wherein a desalting agent in step (b) comprises at least one of sodium dihydrogen phosphate, sodium bicarbonate, potassium dihydrogen phosphate, potassium bicarbonate, ammonium chloride, ammonium sulphate, ammonium bromide, ammonium phosphate, and ammonium carbonate.

9. The process according to claim 1, wherein a desalting agent in (b) comprises an aqueous solution of a salt.

10. The process according to claim 1, wherein a desalting agent in (b) comprises a cation exchange resin, an anion exchange resin, or a chelated resin.

11. The process according to claim 1, wherein a salt in (c) comprises an organic amine salt or a metal salt.

12. The process according to claim 1, wherein a salt in (c) comprises a salt with at least one of dicyclohexylamine, dipropylamine, diisopropylamine, α-methylbenzylamine, cyclohexylethylamine, and t-butyl amine.

13. The process according to claim 1, wherein a salt in (c) comprises a lithium, sodium, potassium, cesium, magnesium, calcium, or strontium salt.

14. The process according to claim 1, wherein a desalting agent in (d) comprises a salt, an organic acid, an inorganic acid, or a resin.

15. The process according to claim 1, wherein a desalting agent in (d) is used in the form of an aqueous solution.

16. The process according to claim 1, wherein a desalting agent in (d) comprises at least one of sodium dihydrogen phosphate, sodium bicarbonate, potassium dihydrogen phosphate, potassium bicarbonate, ammonium chloride, ammonium sulphate, ammonium bromide, ammonium phosphate, and ammonium carbonate.

17. The process according to claim 1, wherein a desalting agent in (d) comprises at least one of acetic acid, oxalic acid, tartaric acid, n-propionic acid, isopropanoic acid, n-butyric acid, and isobutyric acid.

18. The process according to claim 1, wherein a desalting agent in (d) comprises at least one of hydrochloric acid, hydrobromic acid, hydroiodic acid, hydrofluoric acid, sulfuric acid, nitric acid, phosphoric acid, and polyphosphoric acid.

19. The process according to claim 1, wherein a desalting agent in (d) comprises sodium dihydrogen phosphate, ammonium chloride, ammonium sulphate, ammonium bromide, ammonium phosphate or ammonium carbonate.

20. The process according to claim 1, wherein a desalting agent in (d) comprises a cation exchange resin, an anion exchange resin, or a chelated resin.

21. The process according to claim 1, wherein a pharmaceutically acceptable salt of montelukast acid is prepared.

22. The process according to claim 1, wherein a sodium salt of montelukast acid is prepared.

23. Montelukast acid or its salt obtained according to claim 1, having a purity greater than about 99 percent by weight, by high performance liquid chromatography.

24. Montelukast acid or its salt obtained according to claim 1, having a purity greater than about 99.5 percent, by high performance liquid chromatography.
